(19) United States
(12) Patent Application Publication
(10) Pub. No.: US 2022/0400995 A1
(43) Pub. Date: Dec. 22, 2022

(54) RHYTHMIC STIMULUS TO ESTIMATE AN INTRINSIC FREQUENCY OF AN EEG BAND

(71) Applicant: Wave Neuroscience, Inc., Newport Beach, CA (US)

(72) Inventors: Alexander Ring, Newport Beach, CA (US); James William Phillips, Fountain Valley, CA (US); Robert Isenhart, Orange, CA (US); Alfred Jennings Walke, Encinitas, CA (US); Erik Won, Fullerton, CA (US)

(73) Assignee: Wave Neuroscience, Inc., Newport Beach, CA (US)

(21) Appl. No.: 17/662,635

(22) Filed: May 9, 2022

Related U.S. Application Data
(60) Provisional application No. 63/186,012, filed on May 7, 2021.

(51) Int. Cl. A61B 5/16; A61B 5/00

(52) U.S. Cl. CPC A61B 5/165; A61B 5/6803; A61B 5/7257

(57) ABSTRACT

The invention allows an accurate and automated method and system for determining an intrinsic frequency of an EEG band of a person. Intrinsic frequency values (specifically the intrinsic alpha frequency (IAF)) are used to diagnose mental disorders and detect brain anomalies in a person. At present, these estimates are inaccurate for the population that has EEG with low energy in the EEG band. By combining the EEG recording with a stimulus (e.g., light, sound, touch, etc., or a combination), it is possible to determine the IAF, due to the resonant properties of the brain.

### CROSS REFERENCE TO RELATED APPLICATIONS

This application claims the benefit of U.S. Provisional Patent Application Ser. No. 63/186,012, filed on May 7, 2021, entitled “RHYTHMIC STIMULUS TO ESTIMATE AN INTRINSIC FREQUENCY OF AN EEG BAND,” which is hereby incorporated by reference in its entirety.

### BACKGROUND

Over the years, magnetic field treatment has grown in popularity as a method of treating physical and mental disorders. As popularity for this type of treatment grows, it has shown that applying alternating magnetic fields at specific frequencies upon a user produced therapeutic and advantageous effects.

Currently, alternating magnetic field treatments such as Repetitive Transcranial Magnetic Stimulation (rTMS) use an electromagnet that generates a series of alternating magnetic field pulses. These magnetic fields may be applied to a patient at a specific frequency. For example, the follow patents and patent publications describe systems and methods for generating and/or applying a magnetic field to a patient: U.S. Pat. Nos. 9,015,057; 8,480,554; 8,926,490; 8,585,568; 9,446,259; 10,065,048; 8,456,408; 9,713,729; 8,870,737; 8,475,354; 8,888,672; 8,888,673; 8,961,386; 9,308,387; 9,272,159; 9,649,502; 9,962,555; 10,350,427; 9,308,385; 10,029,111; 10,342,986; 10,398,906; 10,420,953; 10,420,482; US 2017/0281035; US 2017/0296837; US 2018/0104504; US 2016/0045756; and US 2018/0229049, each of which is incorporated by reference in their entirety herein.

Many of the methods and systems provided administer a magnetic field at the intrinsic frequency of an EEG band of the patient. Applying a magnetic field at this frequency requires estimation of the intrinsic frequency of the EEG band. The standard technique of finding the intrinsic alpha frequency (IAF) traditionally involves recording EEG while the subject is resting, alert with eyes closed (Valipour, 2014). The IAF is found as the peak of the Fast Fourier Transform (FFT) between 8-13 Hz, although it may alternately be defined as the peak between 8-12 Hz. Alpha activity is often dominant in the posterior region of the brain, such that it is generally referred to as the posterior dominant rhythm. It is often highly symmetrical.

In many cases, the energy of the EEG at that frequency is not significantly above the background neuronal activity in the band, making the estimation of that frequency difficult to accomplish, either through observation or through a detection algorithm.

Knowledge of the intrinsic frequency is valuable for a number of reasons, comprising diagnosis of mental disorders, such as dementia, MCI, or Alzheimer's, estimating perception, attention, concentration, and alertness, and memory. Therefore, it is advantageous to define a method and/or device which allows a user such as an EEG technician or physician to accurately determine an intrinsic frequency of an EEG band, even for EEGs in which the signal level of therhythmic activity at the intrinsic frequency is low.

Rhythmic photic stimulation has been shown to have an effect on brain activity, implemented by flickering or flashing lights within specific frequency ranges. Specifically, flickering photic stimulation at a person's IAF has been shown to alleviate pain or stress and may also improve behavioral performance (Kim, 2016)(Nomura, 2006)(Williams, 2006). Flickering lights tend to trigger EEG activity at frequencies at or near the photic stimulation frequency, resulting in resonant peaks in the EEG band at the stimulation frequency (Fedotchev, 1990). In addition, peaks in the EEG spectra are sometimes found at slightly higher frequencies and sometimes at subharmonic frequencies, but these are generally far lower than the peak of the dominant EEG waveform in the band (Sato, 1961). The brain's reaction as seen in the EEG to photic stimulation in the alpha range will often generate an increased rhythmic response within the alpha EEG range (Scheuler, 1983). Rhythmic stimulation at alpha frequencies has also beenknown to produce antidepressant-like effects (Kim, 2016). Full citations for the references cited in the foregoing parentheticals are found below.
Kim, S., Kim, S., Khalid, A., Jeong, Y., Jeong, B., Lee, S.-T., Jung, K.-H., Chu, K., Lee, S. K., & Jeon, D. (2016). Rhythmical photic stimulation at alpha frequencies produces antidepressant-like effects in a mouse model of depression. PLoS ONE, 11(1), Article e0145374.
Sato, K., Sonoda, T., Nishikawa, T., & Mimura, K. (1961) Some observations on EEG response to photic flicker stimulation. Acta Med. Nagasaki, 5, 188-196.
Nomura T, Higuchi K, Yu H, Sasaki S, Kimura S, Itoh H, et al. (2006) Slow-wave photic stimulation relieves patient discomfort during esophagogastroduodenoscopy. J Gastroenterol Hepatol 21: 54-58. pmid:16706812,
Williams J, Ramaswamy D, Oulhaj A (2006) 10 Hz flicker improves recognition memory in older people. BMC Neurosci 7: 21. pmid:16515710.
Fedotchev A I, Bondar A T, Konovalov V F. Stability of resonance EEG reactions to flickering light in humans. Int J Psychophysiol. 1990 September; 9(2):189-93. doi: 10.1016/0167-8760(90)90073-m. PMID: 2228753.
Scheuler W. Zur klinischen Bedeutung der gesteigerten Photostimulationsreaktion im alpha-Frequenzbereich [Clinical significance of increased reaction to photostimulation in the alpha frequency range]. EEG EMG Z Elektroenzephalogr Elektromyogr Verwandte Geb. 1983 September; 14(3):143-53. German. PMID: 6414803.

### SUMMARY

The present disclosure relates to magnetic field treatment technology. Specifically, to magnetic stimulation including the application of a magnetic field at a patient's intrinsic frequency of an EEG band.

The various embodiments of the present magnetic stimulation apparatus and method have several features, no single one of which is solely responsible for the desirable attributes provided herein. Without limiting the scope of the present embodiments as expressed by the claims that follow, the more prominent features will be discussed briefly. After considering this discussion, and particularly after reading the section entitled “Detailed Description,” one will understand how the magnetic stimulation apparatus and method of the present embodiments can be used in various combinations to provide the advantages described herein.

In an exemplary embodiment, a magnetic stimulation apparatus and methods include systems and methods for determining a patient's intrinsic frequency within an EEG band.

Exemplary embodiments include systems for and methods of determining an intrinsic frequency of an EEG band of a brain of a person having a set of possible intrinsic EEG values of the EEG band comprising: (1) selecting a stimulation frequency equal to an initial value; and (2) providing a rhythmic stimulus at or near the stimulation frequency; and (3) recording an EEG of the brain of the person for a period of time while the rhythmic stimulus is being provided at the stimulation frequency; and (4) calculating a metric of the EEG at the stimulus frequency value; and (5) providing the rhythmic stimulus at or near an updated stimulation frequency; and (6) recording an EEG of the brain of the person for a period of time while the rhythmic stimulus is being provided at the updated stimulation frequency; and (7) calculating a metric of the EEG at the updated stimulation frequency; and (8) repeating steps 5-7 at an updated stimulation frequency that corresponds with a desired set of possible intrinsic EEG values of the EEG band to be tested. The method may also include determining the intrinsic frequency of the patient by selecting the intrinsic frequency of the EEG band as the frequency value where the metric is optimized.

Exemplary embodiments include systems and methods for determining an intrinsic frequency of an EEG band of a brain of a person comprising: (1) a device designed to provide a stimulus to the person; and (2) an EEG recording device capable of recording the person's EEG; and (3) a interface for presenting the intrinsic frequency to a user; and (4) a processor with memory having instruction that when executed by the processor calculates a metric of the person's EEG. The system is configured such that the patient's EEG is recorded while the stimulus is provided to the person and a metric of the patient's EEG is calculated for the EEG as the stimulus is provided at the simulation frequency. In an exemplary embodiment, the system is configured such that the stimulation frequency sequences through a set of frequency values, and the intrinsic frequency is selected as equal to the stimulation frequency where the metric of the EEG recording is optimized.

In an exemplary embodiment, the stimulation frequency and all updated stimulation frequencies used to provide the rhythmic stimulus make up a set of stimulation frequencies. The patient's intrinsic frequency and/or an estimate of the patient's intrinsic frequency is a frequency from the set of stimulation frequencies. The patient's intrinsic frequency and/or an estimate of the patient's intrinsic frequency may be determined as between two frequencies of the set of stimulation frequencies.

In an exemplary embodiment, the metric is the maximum energy of the patient's EEG or a function related to the patient's EEG at the stimulation frequency. The metric may be taken from one or more channels of the EEG. The metric may be optimized when the metric is a maximum compared to the metric calculated at each of the set of stimulation frequencies. The optimization of the metric when more than one EEG channel is considered may include a maximum across a subset of all EEG channels. In other words, the optimization may be determined when a maximum number of EEG channels has a maximum at the stimulation frequency. The optimization may be determined when a subset of EEG channels have a maximum at the stimulation frequency. The optimization may be determined when any one EEG channel has a maximum at the stimulation frequency. The maximum metric may be the amplitude of a signal at the stimulation frequency as compared to the amplitude of a signal another stimulation frequencies. The other stimulation frequencies may include the set of stimulation frequencies.

In an exemplary embodiment, the metric is the average energy of the patient's EEG or a function related to the patient's EEG across a subset of or all of the EEG channels at the stimulation frequency. The metric is optimized when the metric is a maximum compared to the metric calculated at each of the set of stimulation frequencies.

In an exemplary embodiment, the metric is the average bandwidth of the patient's EEG in an area around the stimulation frequency across one or more channels of the patient's EEG and/or as an average of a subset of all EEG channels, and the metric is optimized when the metric is a minimum compared to the metric calculated at each of the set of stimulation frequencies.

In an exemplary embodiment, the metric is the standard deviation of the frequency corresponding to the peak magnitude of the FFT in a range around the stimulation frequency across one or more EEG channels or as a subset of EEG channels or across all EEG channels, and the metric is optimized when the metric is a minimum as compared to the metric calculated at each of the set of stimulation frequencies.

Exemplary embodiments may include a method and device where a rhythmic stimulus is provided while EEG is being recorded. Exemplary embodiments may be used to identify an intrinsic frequency of an EEG band for the person. The rhythmic stimulus results in increased rhythmic activity in the person's EEG at the rhythmic stimulation frequency. This increased activity is especially pronounced when the rhythmic stimulation frequency matches, or is a harmonic or subharmonic of, the intrinsic frequency of the EEG band. The rhythmic stimulus is provided at frequencies across the EEG band (for example, sweeping the stimulation frequency from low to high across the band continuously or at stepped intervals). The intrinsic frequency of the EEG band may be estimated as the stimulation frequency value where the energy of the EEG is maximum. The rhythmic stimulus may be light, sound, touch, vibration, air pressure, or any other stimulus which may affect the EEG band whether it be felt, sensed, not sensed, either consciously or unconsciously, by the person.

### BRIEF DESCRIPTION OF THE DRAWINGS

FIG. 1 shows one example of the method in which a light is flashed at a Flashing Light Frequency (FLF), and the EEG band is the alpha band.

FIGS. 2a-g show an example FFT of a User with light stimulation at incrementing frequencies.

FIGS. 3a-g shows another example of light stimulation at various FLF values in order to estimate the IAF.

FIG. 4 shows an example system of the present invention in which a person wears an EEG cap, which is electrically connected with a processing unit.

FIG. 5 shows an example system of the present invention in which a person wears a headband that comprises two or more EEG electrodes.

FIG. 6 shows an example system of the present invention in which a person wears an EEG headband, which is connected electrically to a processing unit, which comprises an EEG amplifier, a processor, and memory in order to cycle through RSFs in or around the desired EEG band, and to find the RSF which optimizes a function of the EEG recorded while the stimuli is being delivered, and it uses this optimized RSF to estimate the intrinsic frequency of the EEG band.

### DETAILED DESCRIPTION

Example devices, methods and systems are described herein. Any example embodiment or feature described herein is not necessarily to be construed as preferred or advantageous over other embodiments or features. The example embodiments described herein are not meant to be limiting. It will be readily understood that certain aspects of the disclosed devices, systems and methods can be arranged and combined in a wide variety of different configurations, all of which are contemplated herein. Accordingly, any feature, component, concept, or function may be duplicated, removed, combined or otherwise used alone or in combination with any other combination of other features, components, concepts, or functions described herein or otherwise known to a person of skill in the art.

The particular arrangements shown in the figures should not be viewed as limiting. It should be understood that other embodiments might include more or less of each element shown in a given figure. Further, some of the illustrated elements may be combined or omitted. Yet further, an example embodiment may include elements that are not illustrated in the figures.

Exemplary embodiments described herein include a system and method for determining an intrinsic frequency of an EEG band, which comprises recording an EEG of a person while providing a rhythmic stimulus at a specified frequency to the person. The stimulus may be light (flashing light, video, changing colors or intensity, etc.), sound, touch, air pressure, vibration, electric current (transcutaneous, induced electromagnetic, etc.), or any other stimulus which maybe sensed, either consciously or unconsciously, by the person. The stimulus may also be a combination of stimuli. For example, combining light and sound.

Exemplary embodiments of a sensed stimulus may include, for example, a stimulus that is consciously perceived by a patient, through one or more of the patient's senses. Exemplary sensed stimulus may be through tactile sense administered through contact or touch, may be through visual sense such as administered through light or other visual, may be through audio sense administered through sound.

Exemplary embodiments of a stimulus that is not sensed may include, for example, a stimulus that has a stimulus effect on the patient that is unknown to the patient or not received through one of the conventional five senses. A non-sensed stimulus may be a magnetic field, electric field, current, etc.

In an exemplary embodiment, the EEG band may be chosen as one which comprises an intrinsic frequency. For example, the Alpha EEG band may be the EEG band comprising the intrinsic alpha frequency (IAF). Other EEG bands are also possible in the invention. For example, the EEG band may be the Alpha, Beta, Theta, Delta, Gamma, Mu, or other EEG band.

The patient should preferably be calm and relaxed when the rhythmic stimulus is generated. If the stimulus is a flashing light, it should preferably be placed where the person can sense when the light flashes. For example, a strobe light may be placed within the person's field of vision. If the stimulus is sound, the person could wear headphones and listen for an audible beep, warble, or other rhythmically varying audible signal. If the stimulus is touch, then the person could wear a band that comprises a diaphragm, which presses on the person's skin in a rhythmic manner.

If the EEG band is the alpha band, then preferably the person will keep their eyes closed because the intrinsic alpha frequency (IAF) may be most evident on an EEG when the eyes are closed. However, eyes closed is not required to influence the alpha band. If the eyes are closed, then a flashing light should be strong enough so that it can be sensed by the eyes through closed eyelids.

The rhythmic stimulus may cause increased EEG energy at the rhythmic stimulus frequency (RSF), resulting in a visible bump, or local maximum, at this frequency in the magnitude of the Fast Fourier Transform (FFT) of the EEG signal. The increased energy may be particularly evident when the RSF matches the person's IAF.

In the present invention, the EEG is recorded while a rhythmic stimulus is provided at an RSF, and this RSF is shifted periodically to different frequencies in or near the EEG band. For example, the RSF may be shifted by small steps through the band, remaining at a particular frequency for a limited period of time before shifting again. In an exemplary embodiment, the RSF may be administered at a set of frequencies in which the set of frequencies is used sequential. The set of frequencies may make up frequencies within the EEG, and may comprise a step interval. The step interval may be equal or unequal between sequential and/or adjacent frequencies. For example, a larger step may be used to obtain a less precise location of the IAF, and a smaller step interval may be used around and/or near an anticipated IAF. The stimulus maybe applied at each frequencies within the set of frequencies for a period of time. The period of time of each stimulus administration at a given frequency may be the same or may be different.

For example, if the EEG band is the alpha band, the RSF may sequence through 8.0 Hz, 8.1 Hz, 8.2 Hz, . . . , 12.9 Hz, 13.0 Hz, spending 9.0, 10.0, 11.0 . . . seconds at each RSF before shifting to the next in the sequence. Once the RSF steps all the way through a range which comprises the EEG band, then the EEG segments which make up the entire EEG recording may be categorized based upon the RSF of the stimulus that was present during each segment.

In an exemplary embodiment, a metric is obtained for each EEG segment associated with the administration of the stimulus at each of the rhythmic stimulus frequencies. The intrinsic frequency of the EEG band is estimated as the RSF associated with the EEG segment where the metric is optimized.

Exemplary embodiments may obtain and/or use different metrics in order to determine the estimate of the intrinsic frequency. The optimization to assess the metric in order to determine the intrinsic frequency may depend on the metric. For example, the metric may be an amplitude, bandwidth, or other attribute of the energy of the EEG segment. The metric may be determined (such as averaged) across one or more channels of an EEG segment. The energy of the EEG segment may be Fast Fourier Transform (FFT) of the EEG signal.

For example, the metric could be the energy of the FFT of the EEG segments at the RSF. In this case, the energy could be represented by the magnitude of the FFT of the EEG segments at the RSF. If multiple channels of EEG are used, then the energy could be the average magnitude of the FFT of the EEG segments at the RSF across all channels (or a subset of channels of the EEG). In an exemplary embodiment, the energy could be the maximum magnitude of the FFT of the EEG segments at the RSF across all channels (or a subset of channels of the EEG). In an exemplary embodiment, the energy could be the average magnitude of the FFT of the EEG segments at the RSF in a subset of channels of the EEG segment.

In an exemplary embodiment, the optimal metric is the one where the energy of the FFT of the EEG segments at the optimized RSF is maximized as compared to the energy of the FFT of the EEG segments at other RSF frequencies.

In an exemplary embodiment, the metric may be represented by the energy at frequencies outside of the RSF frequency, or outside of a frequency envelope corresponding to the RSF. The frequency envelope may be determined based on the stepwise distance between frequencies used as the RSF. The frequency envelope may also be determined based on standard practice or understanding of a variation or range that achieves the objective of the instant analysis. For example, a frequency envelope may be determined by a standard deviation or a multiple of the standard deviation of the maximum energy at the RSF. For example, if the RSF was presented at 9.0 Hz, the energy as represented by an FFT or measured via other means for frequencies not corresponding to 9.0 Hz or not corresponding to 8.5-9.5 Hz may be measured. An optimal metric in this embodiment may be where the energy is minimized outside of the RSF frequency or RSF frequency envelope as compared to other RSF frequencies.

In an exemplary embodiment, EEG network connectivity measures may be used as an optimal metric, both regional and global. For example, coherence, a measure that compares signal variability between two signals for a given frequency range, and provides a measurement from 0 (no similar information in the signal) to 1 (identical information in the signal). A coherence measurement for EEG may be produced for each inter-electrode pair, so a 19-lead EEG would produce a 19×19 matrix of coherence values, which may correspond to regional (all electrodes in the frontal region) or global (a frontal lead versus a posterior lead) measures, and the average of regional or global electrode pairs at a frequency around the RSF may be used as the optimal measure for ideal subject RSF. In this embodiment, the metric may be optimized when the metric is a maximum at a specific RSF as compared to the metric determined at other RSF outside of the specific RSF.

In an exemplary embodiment, the metric may be the bandwidth of the FFT of the EEG around the RSF. For example, the bandwidth of the FFT of the EEG around the RSF may be lower when the RSF matches the intrinsic frequency of the EEG band. The intrinsic frequency of the EEG band could be estimated as the specific RSF where the bandwidth of the FFT of the EEG segment is lowest as compared to the bandwidth of the FFT of the EEG segment when stimulated at other RSF outside of the specific RSF.

In an exemplary embodiment, the metric may be the variance between channels (all channels or a subset of channels of the EEG band) of the frequency of the peak magnitude of the FFT of the EEG in the EEG band. The peak magnitude of the FFT of the EEG in an EEG band has a lower variance between all channels (or a subset of channels) when the RSF matches the intrinsic frequency of the EEG band. In this case, the metric may be optimized and the intrinsic frequency estimated as the RSF where the standard deviation of the peak values of the magnitude of the FFT of the EEG segment across all channels (or a subset of channels) is minimized.

It has been shown that following light stimulation at RSF that EEG entrainment may occur at the frequency of RSF. In this case, the amplitude, connectivity, and frequency components of the EEG segment following RSF stimulation may constitute the measurement for RSF selection, based on maximization of the parameter versus other RSF frequencies.

It is known that resting EEG characteristics are variable in a population, as such, response on individually selected measurements may also be variable. An ideal measurement may be represented by a multivariate, which is comprised of at least two, if not more previously described metrics, where the included metrics are weighted and the maximization (or minimization) of the resultant multivariate is representative of RSF for estimating the intrinsic frequency of the patient. For example, a subject may have minimal to moderate response for amplitude of EEG band corresponding to the RSF, but maximal response for regional coherence and for reduction in standard deviation of peak values, in which case the resultant multivariate would be maximized relative to other RSF multivariate values, and be selected. Accordingly, exemplary embodiments may include repeating the process across one or more metrics, combining the metrics (such as for example as an average or weighted average), and conducting an assessment on the combined metrics to optimize the combined metric.

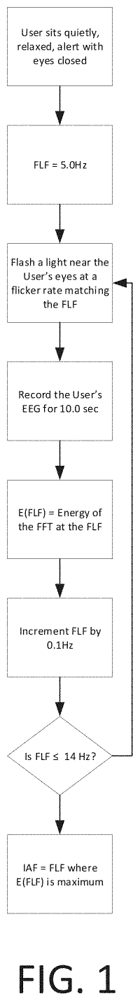

FIG. 1 shows one example of the method in which a light is flashed at a Flashing Light Frequency (FLF), and the EEG band is the alpha band. Therefore, the intrinsic frequency in this example is the Intrinsic Alpha Frequency (IAF). The User sits quietly, relaxed, alert with eyes closed (100). The User preferably has their eyes closed when the EEG band is the alpha band. Visual processing is primarily performed in or near the occipital lobe, which is the region of the brain most likely to generate activity in the alpha EEG band. Closing the eyes allows the alpha activity to be more evident. Optionally, it is also possible to generate activity when the User stares vacantly into space without focusing on any specific object. However, the state whereby alpha EEG is most prevalent is much easier for the User to achieve simply by closing the eyes. In this example, The User could sit in a chair, or recline. Optionally, the User could lie on a flat surface, such as a couch or a bed. The person preferably would be relaxed, because a relaxed state is more likely to result in the brain generating neuronal activity in the alpha EEG band. Also, relaxing keeps the muscles from contracting, which may cause artifacts to appear on the EEG that are independent of brain activity.

In the example, the FLF is initially set to 5.0 Hz. This starting frequency value is well outside the alpha EEG band (101), which is generally defined to be in the range between 8-13 Hz. In this example, the initial FLF is set lower than the lowest frequency of the alpha EEG band. Alternately, the FLF could be set higher than the highest frequency of the alpha EEG band. For example, the initial FLF could be set to 16.0 Hz. Alternately, the initial FLF could be set to a frequency at or near the lowest frequency of the alpha EEG band. For example, the initial FLF could be set to 8.0 Hz. Alternately, the initial FLF could be set to a frequency at or near the highest frequency of the alpha EEG band. For example, the initial FLF could be set to 13.0 Hz.

A light may be flashed with a flicker rate that matches the FLF (102). The light may be flashed close to the User's eyes, or may be above the User's head, or the flashing light may be the only light in an enclosed room. In any case, the system may be configured so that the eyes of the User perceive the flashing light. In general, a User's eyes may still perceive a flashing light even if the eyes are closed, because the light may penetrate the lids.

In this example, a light is flashed. However, the most important factor in affecting neuronal activity is that an external stimulus is provided at the FLF. For example, a sound could be provided which warbles or beeps at a frequency matching the FLF. Alternately, a tapping sensation, such as by a diaphragm, would be provided on the User's skin, where the tapping frequency matches the FLF. Other or additional alternatives, may include a mild electric pulse that may be given at the FLF, which is sensed by the brain.

The User may be aware of the stimulus, or the user may be unaware of the stimulus. For example, if the stimulus level is very low, it may influence the EEG but still be unnoticed by the person. Alternately, the person could be distracted by some means so they are not aware of the stimulus. For example, the User could listen to music. If audio stimulus is administered at a specific frequency and incorporated into the music, it may not be noticed by the User.

The User's EEG is recorded for a period of time. In this example (103), the time is 10.0 seconds. The time could be shorter (for example, 5.0 seconds) or the time could be longer (for example, 30.0 seconds). The period of time should be sufficient so that the EEG recording time so that enough EEG can be recorded to calculate a metric associated with the EEG segment. In this example flowchart, the EEG can be recorded for a length of time to allow the system to measure the energy at the FLF of the magnitude of the FFT of the EEG in the alpha band. If the User has a very clean artifact free EEG, then the EEG recording may be short. However, if the User has an EEG which has significant muscle artifact or is generally low energy or noisy, then a longer recording may be required.

Once the EEG is recorded, a Fast Fourier Transform (FFT) of the EEG waveform may be calculated. Once that is complete, the energy of the FFT at the FLF may be found (104). This energy may be stored in a table, list, array, or other known construct. Once the energy value of the FFT at the FLF is found, the FLF may be incremented. In the example (105), the increment is 0.1 Hz. However, the increment may be smaller in order to obtain a more accurate measure of the intrinsic frequency. For example, the increment may be 0.01 Hz. In this case, the algorithm may take longer to perform. The increment may be larger. For example, the increment may be 0.5 Hz, which could reduce the accuracy of the measurement, but would take less time for the algorithm to perform. Once incremented, then steps 102-105 are repeated.

Once all relevant FLF values have been tested, the algorithm may stop incrementing. For example (106), when the FLF is greater than 14.0 Hz. At this point, the algorithm may select an estimate of the IAF (107). For example, the algorithm may select the intrinsic frequency of the EEG band as the FLF where the energy E(FLF) is maximum.

In the example, the FLF was incremented across the entire EEG band. This may not be required if a prior knowledge exists about possible intrinsic frequency values for the person. For example, if the person has had an intrinsic frequency measurement in the past, the range of values to be checked may be reduced to just encompass a range just around the previously calculated intrinsic EEG value.

The method may also encompass running the method more than once. For example, the system and method may be administered a first time with a greater increment across the entire EEG band, and may then be repeated with a smaller increment in a range around an estimate of the alpha frequency from the first administration at the greater increment. The range for the second administration may be determined, at least in part, on the amount of the greater increment. For example, the range for the second administration may occur one time, two times, three times, four times, or more times of the value of the greater increment above and below the estimate of the alpha frequency from the first administration.

There may be some hysteresis in the EEG of the person in that some of the energy at the previous FLF frequency continues through to the next segment. In order to minimize this hysteresis, a gap in the recording and stimuli may be incorporated. This gap may allow the effects of the previous stimuli to be minimized or removed. Alternately, the intrinsic frequency may be found using a sweep across the band with an incrementing FLF, and compared to the intrinsic frequency that may be found using a sweep across the band with a decrementing FLF. The final intrinsic frequency estimate may be the average between the two intrinsic frequency estimates (when incrementing FLF and decrementing FLF).

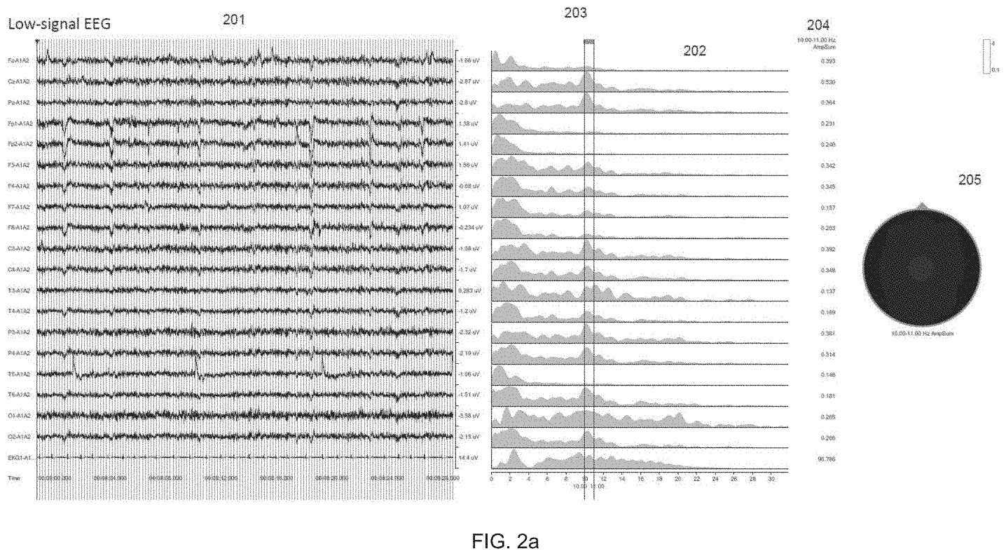

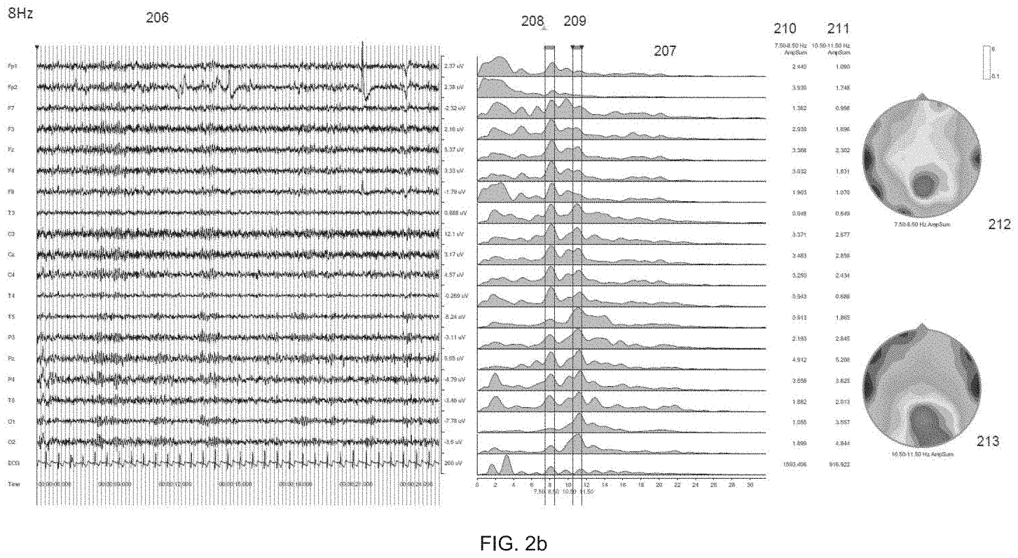

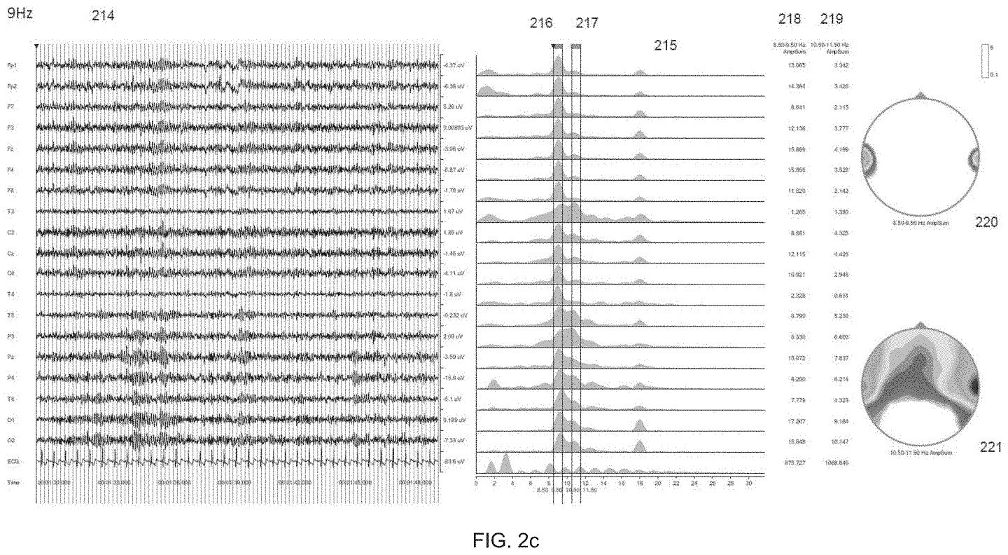

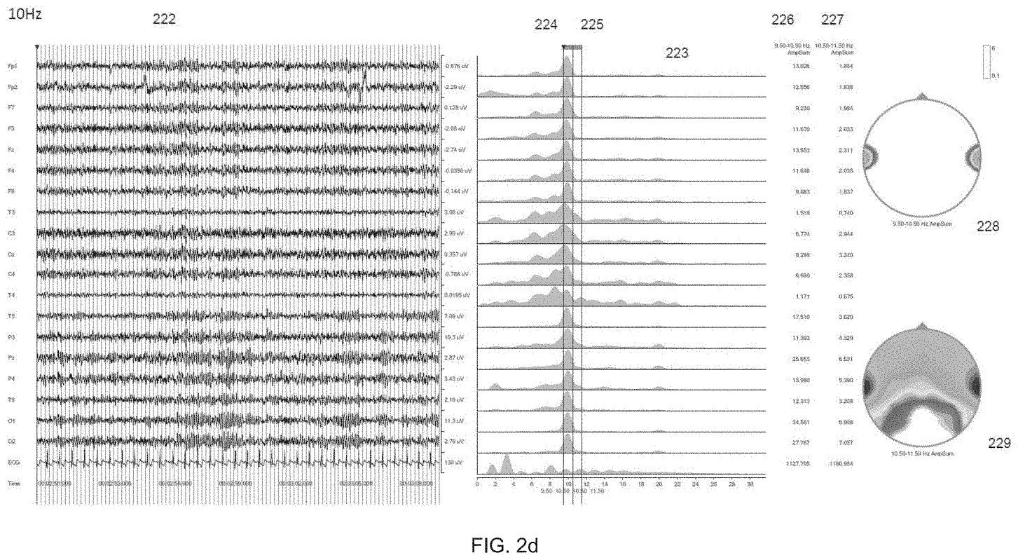

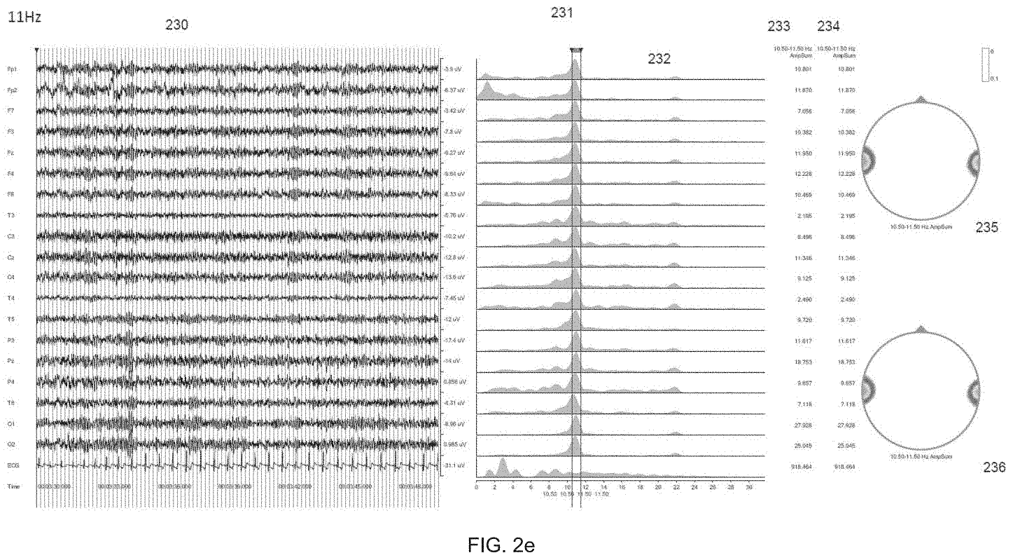

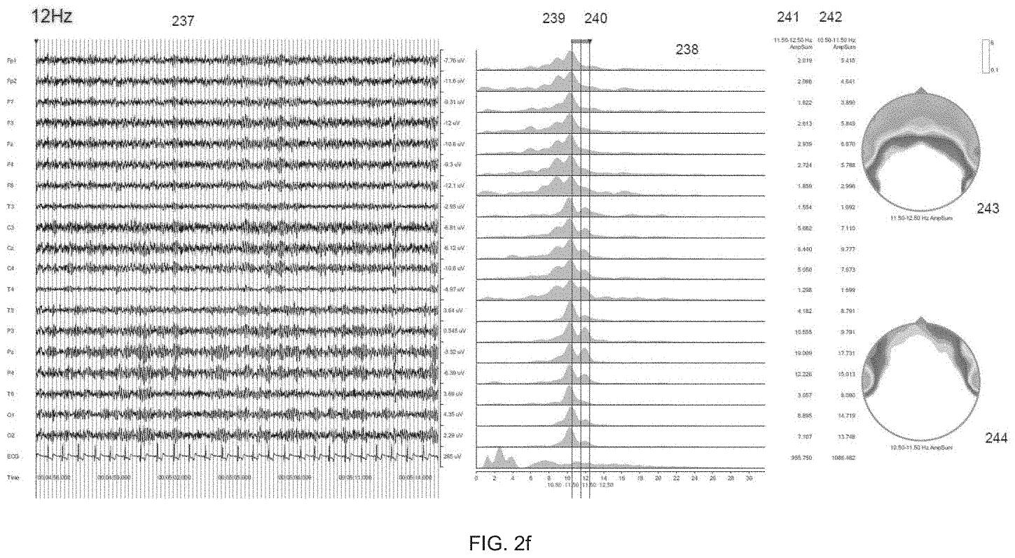

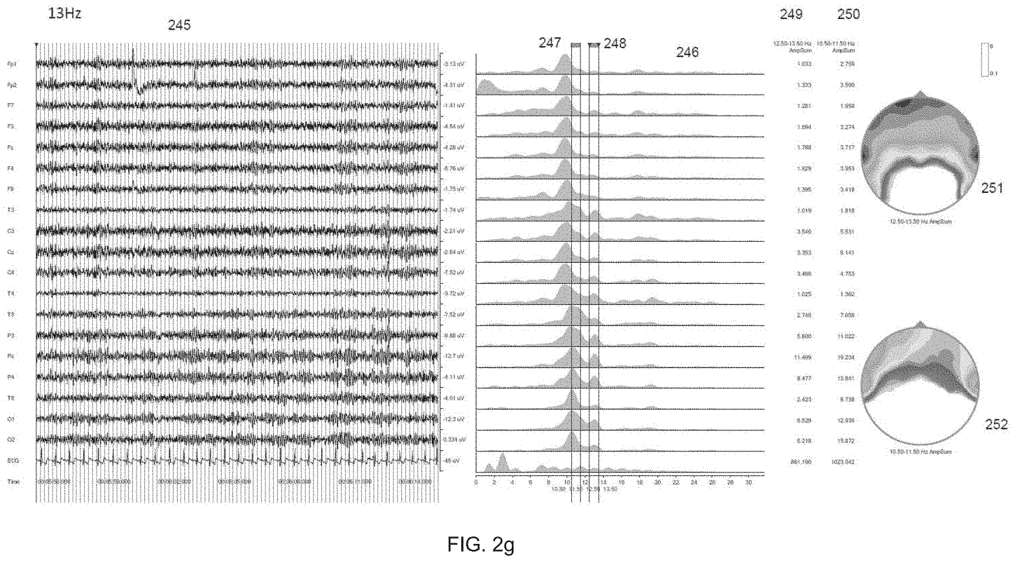

Turning to FIGS. 2a-g, which show examples of EEG and FFT of the EEG from a User according to embodiments described herein with light stimulation at incrementing stimulation frequencies.

In FIG. 2a, the signal strength of the EEG (201) is low, and the energy is distributed across the alpha band, as seen in the FFT of the EEG (202). The EEG is without any external stimulation provided to the User. Conventionally, if the EEG is low energy, the physician is required to interpret the EEG to get an estimate of an intrinsic alpha frequency.

In this example of FIG. 2a, the IAF (203) is estimated at somewhere between 10.0 Hz and 11.0 Hz. It is unclear because there is no clearly defined peak in the FFT that is consistent across electrode locations. The energy near the alpha frequency, as shown by the column (204) varies between 0.146 and 0.530. The energy is widely distributed across the cortex, as shown in the head plot (205).

Having an automated system will make the determination more accurate, more reliable, more repeatable, and faster, allowing the physician better tools for diagnosing and treating mental disorders.

FIG. 2b shows the EEG (206) while a light is flashed near the person's closed eyes at 8 Hz FLF. A peak (208) can be seen in the FFT (207) at 8 Hz, which is comparable to the alpha peak (209), which now appears to be somewhat closer to 11 Hz. The energy of the FFT (211) near the FLF (210) ranges between 1.0 and 3.9, and the energy near the IAF varies between 0.7 and 2.5, which is significantly greater than the energy near the IAF with no stimulation at all in FIG. 2a. The energy of the EEG near the FLF (212) and near the IAF (213) are both concentrated near the back of the brain, which is the location of the occipital lobe that processes light signals.

FIG. 2c shows the EEG (214) with the light flashed at 9 Hz FLF. As can be seen in the FFT (215), a very significant peak (216) has developed at the 9 Hz FLF. The peak (217) near the IAF is still present, but is somewhat overwhelmed by the FLF peak (216). The energy near the FLF (218) now ranges between 1.3 and 15.1. The energy near the IAF (219) ranges between 0.83 and 4.32, which is higher now that the FLF is getting closer. The energy of the EEG near the FLF (220) is now spread across the entire cortex, while the energy near the IAF (221) is still highest near the back of the brain.

FIG. 2d shows the EEG (222) where the FLF is 10 Hz. As can be seen in the FFT (223), the peak at the FLF (224) is even more pronounced, now merging with the peak at the IAF (225). The energy at the FLF (226) now ranges between 1.5 and 34, with the energy at the IAF (227) ranging between 0.7 and 7.0. The energy at the FLF (228) is still distributed across the whole brain, whereas the energy at the IAF (229) is still mainly in the back of the brain.

FIG. 2e shows the EEG (230) with the light flashed at 11 Hz FLF. As the FFT (232) shows, the peak (231) near 11 Hz is focused in a fairly narrow range around the FLF, with very little activity outside this range. The energy (233, 234) is solidly in the range between 2.2 and 27.9 with a narrow bandwidth and little variance between channels, and the distribution (235, 236) is across the entire brain.

FIG. 2f shows the EEG (237) and FFT (238) where the light is flashed at 12 Hz FLF. The peak near the FLF (240) is now clearly more widely spread out, and running into the peak near the IAF (239). The amplitudes of each are fairly equal, ranging between 2 and 19. The distribution of both (243, 244) are significantly higher in the back of the brain.

FIG. 2g shows the EEG and FFT where the light is flashed at 13 Hz FLF. The peak near the FLF (248) continues to diverge from the peak at the IAF (247). The energy of the FFT (250) near the FLF ranges between 1.36 and 11.5, and the energy near the IAF (249) varies between 1.03 and 11.5. The distribution of both (251, 252) are less evenly distributed than the previous frequency shown in FIG. 2f.

From the plots in FIG. 2, the algorithm according to embodiments described herein would estimate an IAF to be between 10 Hz and 11 Hz. The reason is because the average amplitude of the peak is higher across all channels, the bandwidth of the peak is low, and the variance of the peaks between channels is low. Any of these measures, or a combination of some or all, could be used to differentiate and select the correct IAF.

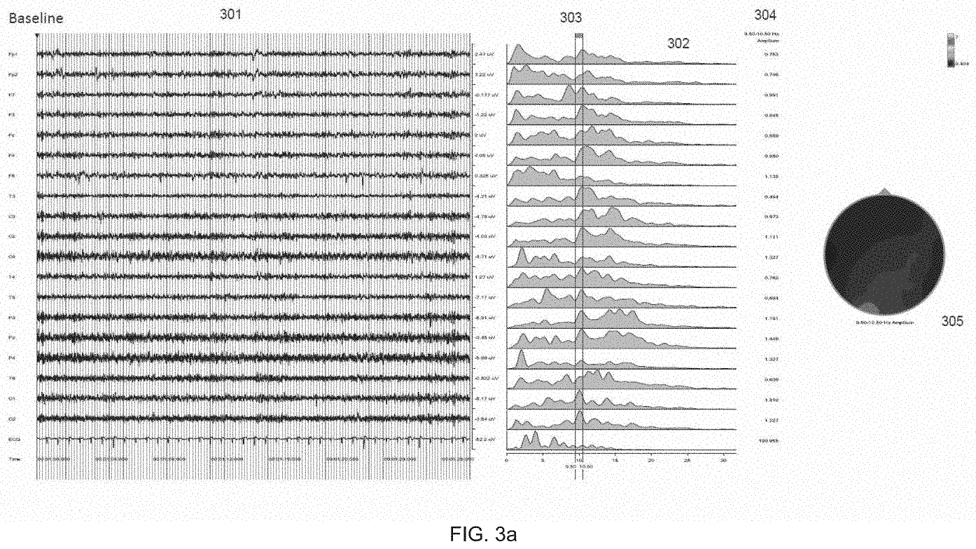

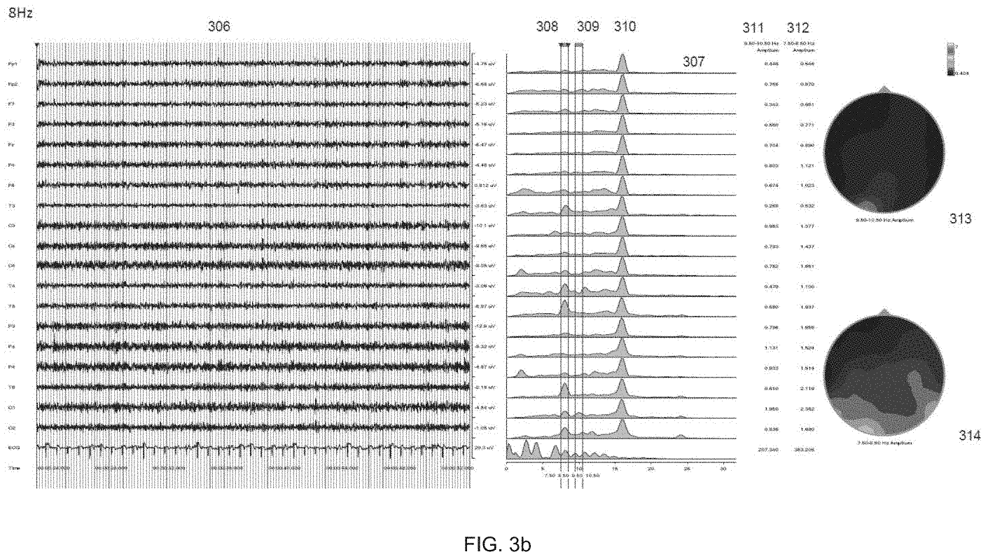

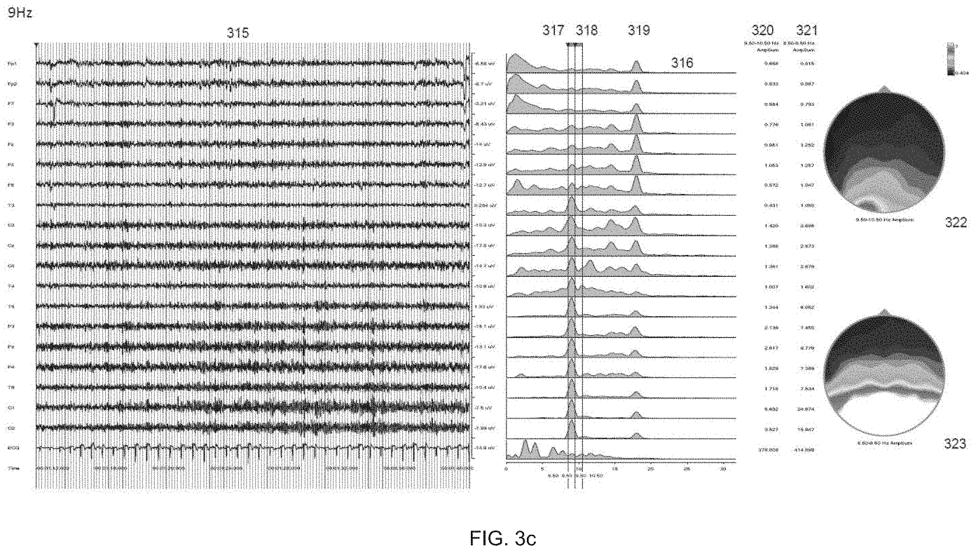

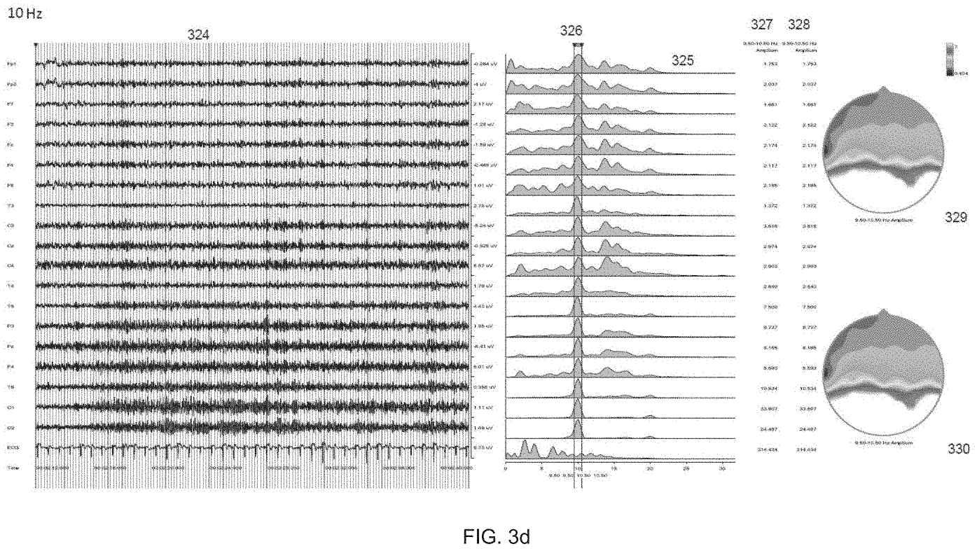

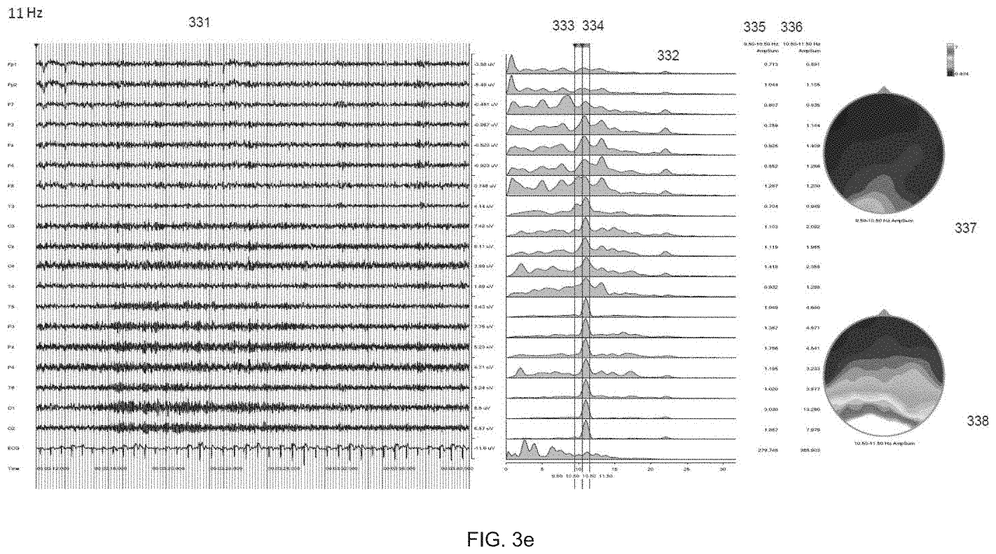

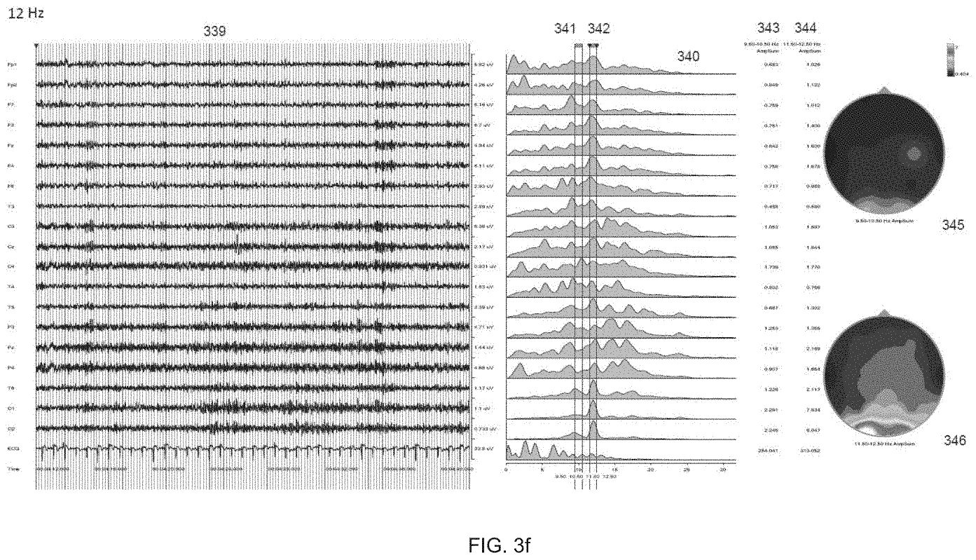

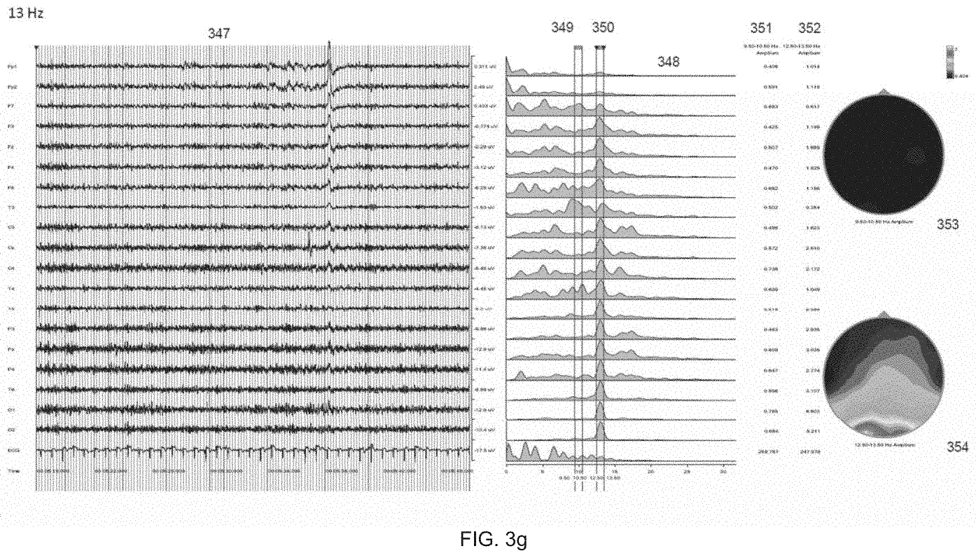

FIGS. 3a-g shows another example of light stimulation at various FLF values in order to estimate the IAF.

FIG. 3a shows the EEG (301) and the FFT (302), which is very low energy with an IAF (303) that likely has very poor accuracy, due to energy all across the alpha EEG band. The energy at the IAF estimate (304) ranges between 0.60 and 1.81. The energy is distributed across the entire cortex.

FIG. 3b shows the EEG (306) and the FFT (307), where a light is flashed at a frequency value of 8 Hz FLF. The peak at the FLF (308) and the estimate of the IAF (309) are both shown, but are overwhelmed by the peak (310) at 16 Hz, which is the 2nd harmonic of the FLF. Preferably, this should be considered an artifact of the stimulation and should not be included in the calculation. The energy around the IAF estimate (311) ranges between 0.28 and 1.95. The energy around the FLF (312) ranges between 0.54 and 2.38. A significant increase from the amplitude with no stimulation. The energy around the IAF estimate (313) is fairly widely distributed, with some focus near the back of the brain. The energy at the FLF (314) is also focused near the back of the brain.

FIG. 3c shows the EEG (315) and FFT (316) when the FLF is 9 Hz. The peak at the FLF (317) and the IAF estimate (318) are starting to merge. A significant peak (319) is still seen at twice the FLF, but it is becoming less of a factor due to the brain's natural frequency response and due to filtering parameters of the EEG amplifier. The energy around the IAF estimate (320) ranges between 0.43 and 2.62, and the energy around the FLF (321) ranges between 0.89 and 7.45. The distribution (322, 323) for both is primarily in the back of the brain.

FIG. 3d shows the EEG (324) and the FFT (325) when the FLF is 10 Hz, which matches the initial IAF estimate. As can be seen by the peak (326), the bandwidth is very low and the variance of the peak frequency is also low. The energy (327, 328) ranges between 1.66 and 33.60, which is the highest of any of the other FLF values. The distribution (329, 330) is primarily in the back of the brain, though the distribution is still fairly wide.

FIG. 3e shows the EEG (331) and FFT (332) when the FLF is 11 Hz, which is above the estimated IAF for the person. Since the peaks at the IAF estimate (333) and the FLF (334) no longer match, the FFT shows that the bandwidth is beginning to increase, with more activity appearing away from the FLF. The energy around the IAF estimate (335) ranges between 0.61 and 3.03 and the energy at the FLF (336) ranges between 0.83 and 13.28. Both are significantly less than the energy levels recorded when the FLF matched the IAF estimate. The distribution for both (337, 338) is primarily in the back of the brain, but beginning to become more widely distributed.

FIG. 3f shows the EEG (339) and the FFT (340) when the FLF is 12 Hz. Since the peaks at the IAF estimate (341) and the FLF (342) are separated more widely, the effect is significantly less, resulting in energy being more widely spread than when the FLF and IAF are closer. The energy at the IAF estimate (343) ranges between 0.47 and 2.29, and the energy at the FLF (344) ranges between 0.59 and 7.83, showing that the overall energy of both peaks is dropping. In addition, the bandwidth is much larger and the variance of the peak value near the FLF is higher. The distribution of energy (345, 346) is still in the back of the brain, but more widely distributed.

FIG. 3g shows the EEG (347) and the FFT (348) when the FLF is 13 Hz. The peak at the IAF estimate (349) is once again very difficult to determine. The peak at the FLF (350) is low with a wide bandwidth, although the bandwidth has decreased somewhat because the energy at the IAF is not influencing the spread of energy at the FLF. The energy at the IAF estimate (351) ranges between 0.41 and 0.79, which is very low. The energy at the FLF (352) ranges between 0.35 and 6.6, which is as low as it has ever been. The distribution at the IAF (353) estimate is very wide across the brain. The distribution at the FLF is also widely distributed, but still concentrated at the back of the brain, which is expected since that is where visual processing occurs.

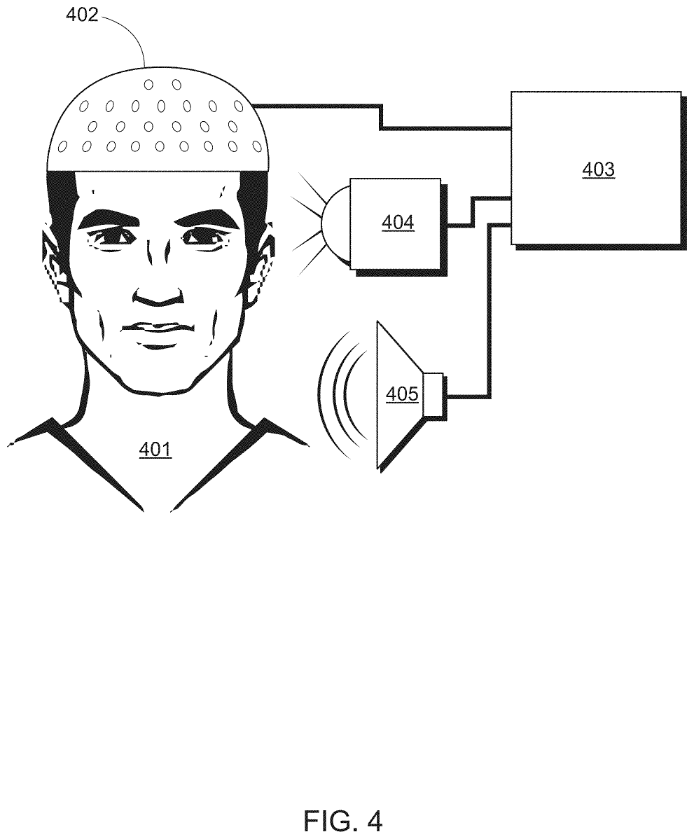

FIG. 4 shows an example system of the present invention in which a person (401) wears an EEG cap (402), which is electrically connected with a processing unit (403). The processing unit comprises an EEG amplifier, a processor, and memory in order to cycle the stimulus through repetitive stimulation frequencies in or around the desired EEG band, and to determine the RSF which optimizes a function (and/or metric) of the EEG recorded while the stimuli is being delivered, and uses that to estimate the intrinsic frequency of the EEG band. The repetitive stimulus in this example is performed using a combination of a flashing light (404) and/or a speaker (405) which plays beeps, ticks, or warbling music at the RSF. The light and speaker may be positioned so that the stimulus can be sensed by the person.

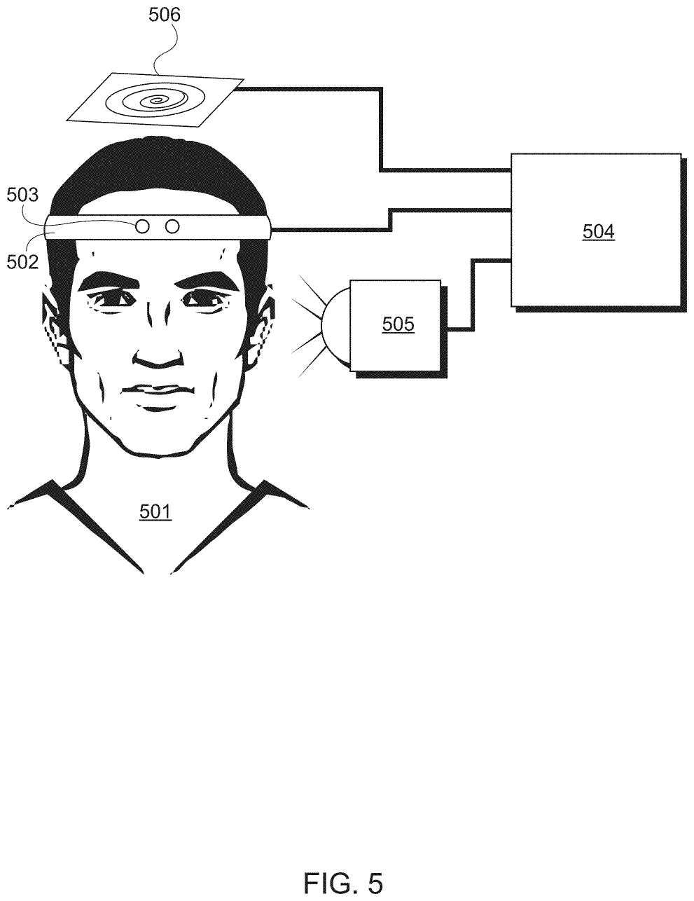

FIG. 5 shows an example system of the present invention in which a person (501) wears a headband (502) that comprises two or more EEG electrodes (503). The EEG electrodes are electrically connected to the processing unit (504), which comprises an EEG amplifier, processor, and memory to cycle through stimulus applied at repetitive stimulation frequencies in and around the desired EEG band, and optimizes a function and/or metric of the EEG while the stimulus is being administered at the RSF across and across a set of RSF values. The estimate of the intrinsic frequency of the EEG band is based upon that optimized metric compared across the stimulus administered at a set of RSF values. The repetitive stimulus in this example is performed using a flashing light (505). The system in this example is combined with a repetitive Transcranial Magnetic Stimulation (rTMS) coil (506), which can stimulate the brain at a pulse frequency based upon the calculated intrinsic frequency of the EEG band. Since the rTMS system also generates a physical sensation as well as brain stimulation, the rTMS pulses themselves may be used as stimuli to affect the EEG of the person, where the rTMS pulses are administered at the RSF.

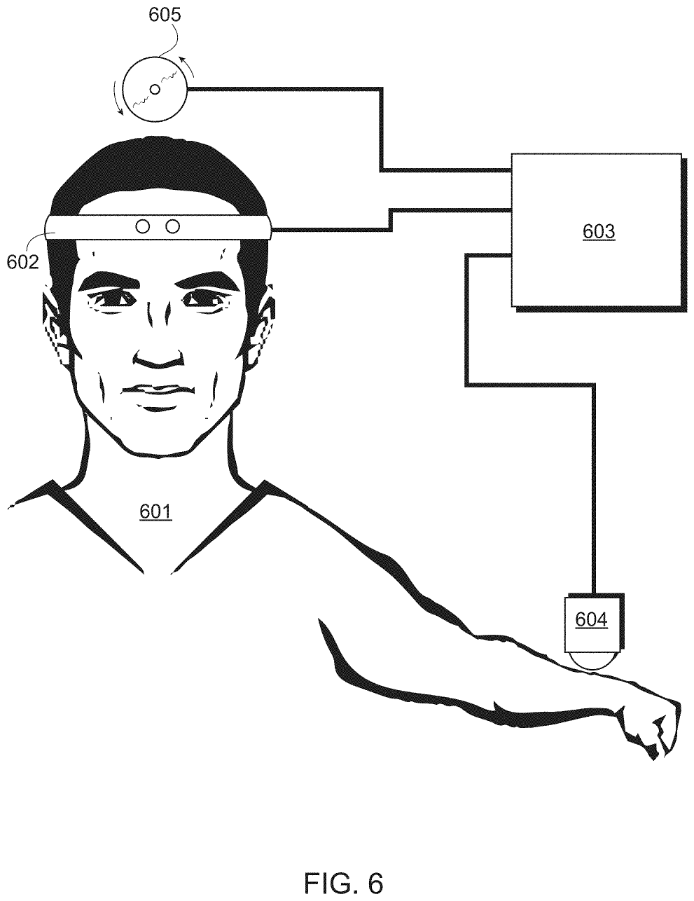

FIG. 6 shows an example system of the present invention in which a person (601) wears an EEG headband (602), which is connected electrically to a processing unit (603), which comprises an EEG amplifier, a processor, and memory in order to cycle the stimulus through RSFs in or around the desired EEG band, and to find the RSF which optimizes a function of the EEG recorded while the stimuli is being delivered, and it uses this optimized RSF to estimate the intrinsic frequency of the EEG band. The stimulus in this exampled is generated using a diaphragm unit (604), which is electrically connected to the processing unit, and where the diaphragm makes physical contact with the skin of the person. In this example, the system is combined with a magnetic field generator (605), which rotates a diametrically magnetized cylindrical magnet near the scalp of the person, in order to provide a low-level alternating magnetic field stimulation of the brain, where the cylindrical magnet is rotated at a frequency based upon the estimated intrinsic frequency of the EEG band.

While the present disclosure has been described with reference to exemplary embodiments, it is to be understood that the invention is not limited to the disclosed exemplary embodiments. The scope of the following claims is to be accorded the broadest interpretation to encompass all modifications, equivalent structures, and functions.

Exemplary embodiments of the system described herein can be based in software and/or hardware. While some specific embodiments of the invention have been shown the invention is not to be limited to these embodiments. For example, most functions performed by electronic hardware components may be duplicated by software emulation. Thus, a software program written to accomplish those same functions may emulate the functionality of the hardware components in input-output circuitry. The invention is to be understood as not limited by the specific embodiments described herein, but only by scope of the appended claims.

As used herein, the terms “about,” “substantially,” or “approximately” for any numerical values, ranges, shapes, distances, relative relationships, etc. indicate a suitable dimensional tolerance that allows the part or collection of components to function for its intended purpose as described herein. Numerical ranges may also be provided herein. Unless otherwise indicated, each range is intended to include the endpoints, and any quantity within the provided range. Therefore, a range of 2-4, includes 2, 3, 4, and any subdivision between 2 and 4, such as 2.1, 2.01, and 2.001. The range also encompasses any combination of ranges, such that 2-4 includes 2-3 and 3-4.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto. For example, any combination of systems and features for applying a stimulus, monitoring the EEG when a stimulus is being applied, and/or the methods and systems for estimating an intrinsic alpha frequency may be used with any system for administering a magnetic field to the patient at the estimated intrinsic alpha frequency. In addition, and combination of stimuli may be used in combination and/or any combination of algorithms to determine one or more metrics and/or optimize the one or more metrics may be used and are within the scope of the instant disclosure.

When used in this specification and claims, the terms “comprises” and “comprising” and variations thereof mean that the specified features, steps, or integers are included. The terms are not to be interpreted to exclude the presence of other features, steps, or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A method of determining an intrinsic frequency of an EEG band of a brain of a person having a set of possible intrinsic EEG values of the EEG band comprising:
(a) selecting a stimulation frequency equal to an initial value;
(b) providing a rhythmic stimulus at or near the stimulation frequency; and
(c) recording an EEG of the brain of the person for a period of time while the rhythmic stimulus is being provided; and
(d) calculating a metric of the EEG at the stimulation frequency; and
(e) if a desired set of possible intrinsic frequency values of the EEG band have not been tested, then selecting an updated stimulation frequency equal to a value in the set of possible intrinsic frequency values of the EEG band which has not been tested and repeating steps (b)-(d) at the updated stimulation frequency; and
(f) estimating the intrinsic frequency of the EEG band as the frequency value where the metric is optimized when comparing the set of metrics calculated from the EEG while the rhythmic stimulus is being provided at each of the set of possible intrinsic frequencies.

2. The method of claim 1 wherein the metric is the maximum energy of the person's EEG at the stimulation frequency across a subset of all EEG channels, and the metric is optimized when the metric is a maximum.

3. The method of claim 1 wherein the metric is the average energy of the person's EEG at the stimulation frequency across a subset of all EEG channels, and the metric is optimized when the metric is a maximum.

4. The method of claim 1 wherein the metric is an average of a bandwidth of the person's EEG in an area around the stimulation frequency across a subset of all EEG channels, and the metric is optimized when the metric is a minimum.

5. The method of claim 1 wherein the metric is a standard deviation of a frequency corresponding to the peak magnitude of the FFT in a range around the stimulation frequency across all EEG channels, and the metric is optimized when the metric is a minimum.

6. A system for determining an intrinsic frequency of an EEG band of a brain of a person comprising:
(a) A device designed to provide a stimulus to the person;
(b) an EEG recording device capable of recording the person's EEG;
(c) an interface for presenting the intrinsic frequency to a User; and
(d) a processor with memory having instruction that when executed by the processor calculates a metric of the person's EEG, wherein the system is configured such that a person's EEG is recorded while the stimulus is provided to the person at a stimulation frequency and the metric of the person's EEG is calculated at the stimulation frequency, and the system repeats the administration of a stimulation, the recording of the EEG while the stimulus is provided, and the calculation of the metric of the person's EEG at each of a set of stimulation frequency values, and the intrinsic frequency is determined as equal to the stimulation frequency where the metric of the EEG recording is optimized.

7. The method of claim 6 wherein the metric is the maximum energy of the person's EEG at the stimulation frequency across a subset of all EEG channels, and the metric is optimized when the metric is a maximum.

8. The method of claim 6 wherein the metric is the average energy of the person's EEG at the stimulation frequency across a subset of all EEG channels, and the metric is optimized when the metric is a maximum.

9. The method of claim 6 wherein the metric is an average of a bandwidth of the person's EEG in an area around the stimulation frequency across a subset of all EEG channels, and the metric is optimized when the metric is a minimum.

10. The method of claim 6 wherein the metric is a standard deviation of a frequency corresponding to the peak magnitude of the FFT in a range around the stimulation frequency across all EEG channels, and the metric is optimized when the metric is a minimum.
